(19) United States
(12) Patent Application Publication
(10) Pub. No.: US 2005/0281758 A1
(43) Pub. Date: Dec. 22, 2005

(54) Oral care compositions

(76) Inventors: Kenneth T. Dodd, Upton, MA (US); Ronald M. Gurge, Franklin, MA (US); David V. Tyndall, Medway, MA (US)

Correspondence Address: FISH & RICHARDSON PC, P.O. BOX 1022, MINNEAPOLIS, MN, 55440-1022, US

(21) Appl. No.: 10/871,659

(22) Filed: Jun. 18, 2004

(51) Int. Cl. A61K 9/16; A61K 7/20

(52) U.S. Cl. 424/49

(57) ABSTRACT

Oral care compositions are described herein. The oral care compositions generally have Theological properties that are desirable for use in a dispensing toothbrush. In some instances, the oral care composition has a yield point of y Pa and a shear slope of x Pa/Sec, wherein the ratio of yield point to shear slope is less than y/(74−370x+470x²). In some instances, the oral care composition has a Newtonian viscosity at 400 Pa of less than 1.0 Pa.s.

### TECHNICAL FIELD

This invention relates to an oral care composition which can be dispensed from a dispensing toothbrush.

### BACKGROUND

The current oral care market presents the consumer with a variety of dentifrice, e.g., toothpaste, options for use on either conventional manual or power toothbrushes. The offerings range from tooth powders to clear gels to the more common opaque pastes. Dentifrice is widely recognized as important in oral health, especially for the reduction of caries. Most compositions contain active ingredients for cleaning the oral cavity, such as abrasives and surfactants, anticavity ingredients, such as fluoride, tartar control ingredients, pH buffers, whitening agents, humectants, and a variety of actives for treating an array of oral health problems. Dentifrice formulations vary tremendously depending upon a variety of factors, including ingredient compatibility, cost, and the desired benefits and quality of the product.

In most instances, a dentifrice is a three-phase system, including a continuous phase, a dispersed phase, and a solid phase. While the appropriate blending and relative composition of these components is important to obtain a stable system, the flow characteristics or Rheology of the formulation is important for consumer acceptance. For example, it is generally desirable that a dentifrice does not separate with aging, is easy to dispense from a toothpaste tube, demonstrates good ribbon properties without strings, holds form on the top of a toothbrush, and distributes evenly and smoothly over the teeth.

### SUMMARY

Oral care compositions such as dentifrice are described below. Generally, the oral care compositions described herein have rheological properties that provide desirable dispensing characteristics when dispensed from a dispensing toothbrush. The inventors have found that, when oral care compositions are dispensed using a dispensing toothbrush, some of the traditional rheological characteristics associated with dentifrice are not necessary. For example, when the oral care composition is delivered while the head of the dispensing toothbrush is in the user's mouth, it is not generally necessary that the oral care composition hold its shape on the brush. On the other hand, it is generally desirable that the oral care composition flow easily through a mechanical pumping system, thus providing a continuous and even dispensing of the oral care composition from the dispensing toothbrush.

The present invention features oral care compositions having rheological properties that render the compositions suitable for use in dispensing toothbrushes. Some preferred compositions also exhibit good sensory characteristics, e.g., flavor intensity and impact, tingle, ease of rinsing and ease of spreading, and good efficacy, e.g., cleaning, tartar removal, whitening and anti-caries protection. The compositions also generally remain stable during aging, e.g., exhibiting little or no phase separation after 3 months at accelerated aging conditions of 40° C. and 45% Relative humidity.

In one aspect, the invention features an oral care composition that includes a carrier, a binder, and an active ingredient. The oral care composition exhibits a yield point of y Pa and a shear slope of x Pa/Sec, where the ratio of yield point to shear slope is less than y/(74−370x+470x²), provided that when the yield point is less than 5 Pa, the shear slope is greater than 0.2 Pa/Sec. The oral care composition can have one or more of the following characteristics.

In some instances, the yield point is greater than 5 Pa.

In some instances, the binder includes at least one of hydrous sodium lithium magnesium silicate or sodium carboxymethylcellulose. For example, the binder can include about 0.1 to about 2.0% by weight of hydrous sodium lithium magnesium silicate or sodium carboxymethylcellulose. Alternatively, the binder can include both hydrous sodium lithium magnesium silicate and sodium carboxymethylcellulose.

In some instances, the binder includes a synthetic clay, for example a laponite, a hectorite, or a betaonite.

In some instances, the oral care composition can also include a surfactant or a detergent. Examples of surfactances include sodium lauryl sulphate, cocamidopropyl betaine, or D-glucopyranoside C₁₀₋₁₆ alkyl oligomeric, or mixtures of one or more of the above.

In some instances, the oral care composition includes a thickening agent. An example of a thickening agent includes thickening silica. In some instances, the oral care composition includes between about 4% to about 8%, for example about 6% thickening silica. In other instances, the thickening agent includes a polymer, for example an alginate, cellulose, a cellulose derivative, or carageenan. In some instances the thickening agent is carageenan. For example, the oral care composition can include between about 0.1% and about 2.0% by weight of carageenan.

In some instances, the oral care composition includes hydrous sodium lithium magnesium silicate, sodium carboxymethylcellulose, sodium lauryl sulphate, cocamidopropyl betaine, and D-glucopyranoside C₁₀₋₁₆ alkyl oligomeric.

In some instances, the oral care composition includes a polishing agent. Examples of polishing agents include sodium bicarbonate, water-colloidal silica, percipiated silicas, sodium aluminosilicate, silica grades containing alumina, hydrated alumina, dicalcium phosphate, insoluable sodium metaphosphate, calcium carbonate, magnesium, trimagnesium phophate or combinations thereof. In some instances the oral care composition includes abrasive silica or hydrated silica.

In some instances the oral care composition includes xanthan gum and hydrous sodium lithium magnesium silicate. For example, the oral care composition can include between about 0.1% to about 2.0% by weight combined of xanthan gum and hydrous sodium lithium magnesium silicate. In some instances, the oral care composition can have a ratio of the xanthan gum to hydrous sodium lithium magnesium silicate of between about 0.5:1 to about 2:1.

In some instances, the active ingredient includes an antimicrobial agent, a phenolic compound, an anti-inflammatory agent, an anti-caries agent, a plaque buffer, a desensitizing agent, an anti-calculus agent, a biomolecule, or combinations thereof.

In some instances, the carrier includes water, polyethylene glycol, polypropylene glycol, glycerin or combinations thereof.

In some instances, the oral care composition has a Newtonian viscosity at 50 to 400 Pa of less than 1.0 Pa.s, for example a Newtonian viscosity at 50 to 400 Pa of less than 0.5 Pa.s.

In some instances, the oral care composition includes a humectant. Examples of a suitable humectant include glycerin, sorbitol or a combination thereof.

In some instances, the oral care composition includes a salt. Examples of salts include sodium saccharin, tetrasodium pyrophosphate, sodium hydroxide and combinations thereof.

The oral care composition can be, for example, a dentifrice.

In another embodiment, the invention includes an oral care composition that has a carrier, and, dispersed in the carrier, sodium lauryl sulphate, cocamidopropyl betaine, and D-glucopyranoside C₁₀₋₁₆ alkyl oligomeric.

In some instances, the oral care composition also includes hydrous sodium lithium magnesium silicate and sodium carboxymethylcellulose.

In some instances, the oral care composition includes about 0.1 to 1.0% hydrous sodium lithium magnesium silicate, about 0.1 to 1.0% sodium carboxymethylcellulose, about 1.0 to 2.0% sodium lauryl sulphate, about 0.2 to 1.2% cocamidopropyl betaine, and about 0.1 to 1.0% D-glucopyranoside C₁₀₋₁₆ alkyl oligomeric. For example, the oral care composition can include about 0.4 to 0.6% hydrous sodium lithium magnesium silicate, about 0.35 to about 0.40% sodium carboxymethylcellulose, about 1.5 to 1.6% sodium lauryl sulphate, about 0.6 to 0.8% cocamidopropyl betaine, and about 0.4 to 0.6% D-glucopyranoside C₁₀₋₁₆ alkyl oligomeric.

In some instances, the oral care composition includes a thickening agent. An example of a thickening agent includes thickening silica, for example about 4% to about 8% thickening silica by weight, for example about about 6% thickening silica by weight. In some instances, the thickening agent is a polymer, for example an alginate, cellulose, a cellulose derivative, or carageenan.

In some instances, the oral care composition includes an abrasive.

In some instances, the oral care composition is a dentifrice.

In another embodiment, the invention includes an oral care composition including a carrier, a binder including at least one of hydrous sodium lithium magnesium silicate or sodium carboxymethylcellulose, and an active ingredient, wherein the oral care composition exhibits a yield point of y Pa and a shear slope of x Pa/Sec, and wherein the ratio of yield point to shear slope is less than y/(74−370x+470x²), provided that when the yield point is less than 5 Pa, the shear slope is greater than 0.2 Pa/Sec.

In some instances, the oral care composition includes both hydrous sodium lithium magnesium silicate and sodium carboxymethylcellulose.

In another embodiment, the invention includes an oral care device that includes a housing including, at a distal portion of the housing, a head dimensioned to fit within a user's mouth; a compressible member defining at least a portion of a fluid path disposed within the housing; a pumping assembly disposed within the housing and configured to compress the compressible member progressively along at least a portion of its length to transfer fluid along the fluid path toward an outlet at the distal portion of the housing; and an oral care composition, disposed within the oral care device, wherein the oral care composition exhibits a yield point of y Pa, and a shear slope of x Pa/sec, and wherein the ratio of yield point to shear slope is less than y/(474−370x+470x²), provided that when the yeild point is less than 5, the shear slope is greater than 0.2 Pa/sec.

In some instances, the oral care composition is a dentifrice.

In another embodiment, the invention includes an oral care device that includes a housing including, at a distal portion of the housing, a head dimensioned to fit within a user's mouth; and a pumping assembly disposed within the housing and configured to transfer an oral care composition along a fluid path from a source containing the composition toward an outlet at the distal portion of the housing; wherein the oral care composition exhibits a yield point of y Pa, and a shear slope of x Pa/sec, and wherein the ratio of yield point to shear slope is less than y/(474−370x+470x²), provided that when the yeild point is less than 5, the shear slope is greater than 0.2 Pa/sec.

In another embodiment, the invention includes a method comprising brushing teeth of a mammal with an oral care composition comprising a yield point of y Pa, and a shear slope of x Pa/sec, wherein the ratio of yield point to shear slope is less than y/(74−370x+470x²), provided that when the yeild point is less than 5, the shear slope is greater than 0.2 Pa/sec. The oral care composition can be, for example a dentifrice.

Another aspect includes using the dentifrice with a toothbrush. Suitable toothbrushes include dispensing power toothbrushes. Some toothbrushes include, for example, (a) an elongated housing including, at a distal portion of the housing, a head dimensioned to fit within a user's mouth; (b) a fluid conduit defining at least a portion of a fluid passageway in the housing, the fluid conduit having a compressible region disposed in the housing; and (c) a motorized pumping assembly configured to compress the fluid conduit in the compressible region progressively along at least a portion of the length of the fluid conduit to draw fluid into the compressible region and to transfer fluid out of the compressible region along the fluid passageway toward an outlet at the distal portion of the housing. The compressible region of the fluid conduit defines a path that is not semi-circular. Preferably the path is also not generally circular. In some implementations, the path defined by the compressible region is substantially straight, i.e., it has a radius of curvature greater than half the diameter of the housing in cross-section. The radius of curvature may in some cases be greater than 2 inches. In other implementations, the path defined by the compressible region may include one or more localized arcuate areas, but in such implementations the path will also include generally linear areas. The geometry of the compressible region allows the pumping assembly to have a relatively small profile, so that the pumping assembly fits within the elongated housing without the diameter of the housing becoming unwieldy. The geometry of the compressible region also facilitates removal of the fluid conduit from the housing. Some toothbrushes may include a reversible pumping assembly configured to transfer fluid along the passageway. The toothbrush may include an energy source for powering the toothbrush, and the housing may include a separable cartridge component that contains a fluid reservoir and the energy source. The toothbrush may include a housing having a movable head configured to rotate about an axis of rotation, a handle and a neck connecting the head and the handle, the housing defining a housing axis extending between the handle and the head that is perpendicular to the axis of rotation, a fluid passageway located within the neck of the housing and extending to an outlet at the head, and a drive member connected to the head at a location spaced from the housing axis, the drive member being configured to rotate the movable head about the axis of rotation. Suitable toothbrushes are described, for example, the following U.S. patent applications: “ORAL CARE DEVICE”, Attorney Docket No. 00216-629001, filed Jun. 3, 2004; “ORAL CARE DEVICE”, Attorney Docket No. 00216-640001, and filed Jun. 3, 2004; “ORAL CARE DEVICE”, Attorney Docket No. 00216-641001, filed Jun. 3, 2004, the entire disclosures of which are incorporated herein by reference.

The invention also features methods of providing oral care using the oral care compositions and toothbrushes described herein. In general, each of the oral care compositions can be used with any of the oral care devices described herein.

The term “Newtonian viscosity” refers to the viscosity of a Newtonian fluid. A Newtonian fluid is one in which the viscosity does not vary with the deformation rate or time. In other words the fluid flow and the rate of shear are generally proportional to the applied stress. Many liquids demonstrate this behavior under a wide range of shear rates while others show this type of behavior under only a limited range of shear rates.

Dentifrice formulations exhibit Bingham plastic behavior where the formulation shows little or no deformation up to a certain level of stress. This yield stress or “yield point” refers to the stress limit at which permanent deformation takes place. The deformation can take place, for example, by undergoing flow.

As viscosity is defined in terms of the shear stress versus the strain rate, the “shear slope” is the rate of change of the viscosity after the fluid begins to flow.

The term “dentifrice” refers to a substance, such as a tooth powder, clear gel or opaque paste, for cleaning teeth.

The term “active ingredient” refers to any material included in an oral care composition that provides a benefit to the user. Examples of such benefits include anti-caries activity, anti-halitosis activity, anti-microbial activity, tooth whitening activity, etc.

Unless otherwise noted, all percentages given are percent by weight.

The details of one or more embodiments of the invention are set forth in the accompanying drawings and the description below. Other features and advantages of the invention will be apparent from the description and from the claims.

### DESCRIPTION OF DRAWINGS

FIG. 1 is a side perspective view of an embodiment of an oral care system.

FIG. 2A is a front perspective view of an embodiment of an oral care device.

FIG. 2B is a rear perspective view of the oral care device of FIG. 2A.

FIG. 3A is a transparent front view of the oral care device of FIG. 2A.

FIG. 3B is a transparent rear view of the oral care device of FIG. 2A.

### DETAILED DESCRIPTION

Oral care compositions such as dentifrice can be dispensed in a variety of ways. For example, dentifrice can be dispensed from a tube, or alternatively, directly from a toothbrush. As discussed above, the inventors have found that, in many instances, the rheological properties that are desirable for a dentifrice dispensed from a tube are different from the properties desirable in a dentifrice dispensed directly from a toothbrush.

Applicants have also discovered combinations of materials that provide oral care compositions having desirable rheological properties, especially when used in combination with a dispensing toothbrush.

The oral care compositions generally include a binder, a carrier, and an active ingredient. In some instances, the oral care compositions also include one or more of the following: a surfactant and/or detergent, a thickening agent, a polishing agent, a carrier, a humectant, a salt, etc.

Binder

The binder system, generally, is a primary factor that determines the rheological characteristics of the oral care composition. The binder also acts to keep any solid phase of the oral care composition suspended in the formulation, thus preventing separation of the solid phase portion of the oral care composition from the liquid phase portion of the formulation. Additionally, the binder can provide body or thickness to the oral care composition. Thus, in some instances, a binder can also provide a thickening function to an oral care composition.

The binder is chosen to provide the oral care composition with shear-thinning properties, i.e., a rapid loss in viscosity as a shear force is applied to the oral care composition, that allow the oral care composition to flow easily through a dispensing toothbrush (e.g., through a mechanical pumping assembly such as a peristaltic pumps). In some instances, a combination of binders are used together to provide a binder system with desirable shear-thinning properties.

Examples of binders that can provide desirable rheological characteristics include sodium carboxymethyl-cellulose, cellulose ether, xanthan gum, carrageenan, sodium alginate, carbopol, hydrous sodium lithium magnesium silicate. In each instance, these binders can also be used in combination binder systems to provide an oral care composition with desirable rheological characteristics.

In one embodiment, the oral care composition has a binder system that includes a combination of hydrous sodium lithium magnesium silicate (commonly referred to as “laponite”) and sodium carboxymethylcellulose (CMC). In some instances, the oral care composition includes at least about 0.15% hydrous sodium lithium magnesium silicate by weight, based on the total weight of the composition (e.g., at least about 0.20%, 0.25%, 0.30%, 0.35%, or 0.40%) and at most about 1% hydrous sodium lithium magnesium silicate (e.g., at most about 0.95%, 0.90%, 0.85%, 0.80%, 0.75%, 0.70%, or 0.65%). The oral care composition also includes at least about 0.08% sodium carboxymethylcellulose (e.g., at least about 0.10%, 0.12%, 0.13%, 0.15%, 0.20% or 0.25%) and at most about 0.65% sodium carboxymethylcellulose (e.g., at most about 0.60%, 0.55%, 0.50%, 0.45%, 0.40%, or 0.35%).

In some instances, thickening silica (e.g., fumed silica) is included in the oral care compositions in addition to the hydrous sodium lithium magnesium silicate and sodium carboxymethylcellulose binding system. For example, the oral care composition can include at least about 2% (e.g., at least about 3%, at least about 4%, at least about 5%, or at least about 6%) and at most about 10% (e.g., at most about 9%, at most about 8%, at most about 7%, or at most about 6%) of a thickening silica.

Preferably, the ratio of hydrous lithium magnesium silicate to sodium carboxymethylcellulose is in the range of about 1:1 to 3:2. If thickening silica is included, the ratio of hydrous lithium magnesium silicate to sodium carboxymethylcellulose to thickening silica can be in the range of about 1:1:16 to 3:3:32.

Table 1 below provides amounts of hydrous sodium lithium magnesium silicate, sodium carboxymethylcellulose, and thickening silica used in certain embodiments of an oral care composition.

TABLE 1
Oral care composition formulations

| | Concentration in |
|---|---|
| Ingredients | percent by weight |
| Hydrous Sodium Lithium Magnesium Silicate | 0.25-0.75 |
| Sodium Carboxymethylcellulose | 0.13-0.50 |
| Thickening Silica | 4-8 |

Oral care compositions having various percent by weight concentrations of hydrous sodium lithium magnesium silicate, sodium carboxymethylcellulose and thickening silica (e.g., SILOX™ fumed silica) were tested to determine rheological properties including the yield point Pa and shear slope Pa/sec⁻¹ as described above. Examples of oral care compositions having various combinations of hydrous sodium lithium magnesium silicate (i.e., laponite), sodium carboxymethylcellulose (i.e., CMC) and SILOX™ silica are shown in Table 2 below together with the yield point and shear slope of each formulation. A preferred yield point would occur between 10 and 80 Pa coupled with a shear slope ranging form 0.06-0.11. For those skilled in the art, the selection of an appropiate range is generally based on the amount of solid phase, the robustness of the pump system and the length and size of the conveyance tubing.

TABLE 2
Rheological Properties of Oral Care Compositions Including CMC,
Laponite, and Silica

| CMC | Laponite | Silica | Yield | Shear Slope |
|---|---|---|---|---|
| (% by wt.) | (% by wt.) | (% by wt.) | point (Pa) | (Pa/sec⁻¹) |
| 0.13 | 0.50 | 4.00 | 1.00 | 0.08 |
| 0.13 | 0.75 | 4.00 | 10.00 | 0.10 |
| 0.25 | 0.25 | 4.00 | 6.31 | 0.13 |
| 0.25 | 0.50 | 4.00 | 25.12 | 0.07 |
| 0.25 | 0.75 | 4.00 | 39.81 | 0.08 |
| 0.38 | 0.25 | 4.00 | 25.12 | 0.09 |
| 0.38 | 0.50 | 4.00 | 31.62 | 0.08 |
| 0.38 | 0.75 | 4.00 | 79.24 | 0.06 |
| 0.50 | 0.25 | 4.00 | 50.12 | 0.07 |
| 0.50 | 0.75 | 4.00 | 79.43 | 0.07 |
| 0.13 | 0.50 | 8.00 | 10.00 | 0.10 |
| 0.13 | 0.75 | 8.00 | 19.91 | 0.11 |
| 0.25 | 0.25 | 8.00 | 25.12 | 0.07 |
| 0.25 | 0.50 | 8.00 | 25.12 | 0.11 |
| 0.25 | 0.75 | 8.00 | 50.12 | 0.06 |
| 0.38 | 0.25 | 8.00 | 50.12 | 0.06 |
| 0.38 | 0.50 | 8.00 | 50.00 | 0.08 |
| 0.38 | 0.75 | 8.00 | 79.24 | 0.06 |
| 0.50 | 0.25 | 8.00 | 63.09 | 0.07 |
| 0.50 | 0.75 | 8.00 | 79.43 | 0.06 |

While binder systems including a combination of hydrous sodium lithium magnesium silicate and sodium carboxymethylcellulose have been described above, other binding systems are also envisioned. For example, in some instances, a combination of xanthan gum with hydrous sodium lithium magnesium is used in the oral care composition. In some instances the total weight of the combination of xanthan gum and hydrous sodium lithium magnesium is about 0.1 to about 2.0% by weight of the oral care composition.

In other instances, carrageenan is used alone or in combination with one or more of the other binders discussed above as a binder in the oral care composition. For example, the oral care composition can include from about 0.05% to about 3% by weight (e.g., from about 0.1% to about 2.0%) of carrageenan.

Other examples of suitable binders include polymers such as hydroxypropyl methylcellulose, hydroxyethyl cellulose, guar gum, tragacanth gum, karaya gum, arabic gum, Irish moss, starch, and alginate. Alternatively, the binder can include a clay, for example, a synthetic clay such as a hectorite, or a natural clay. Each of the binders can be used alone or in combination with other binders.

Surfactants/Detergents

In some instances, the oral care composition (e.g., a dentifrice) includes one or more surfactants or detergents to provide a desirable foaming quality to the oral care composition.

Applicants have discovered that the combination of sodium lauryl sulfate, cocamidopropyl betaine, and D-glucopyranoside C10-C16 alkyl oligomeric provide, give desirable foam characteristics to the oral care composition. In some instances, the oral care composition includes at least about 0.8% sodium lauryl sulphate (e.g., at least about 1.0%, 1.2%, 1.4%, or 1.5%) and at most about 2.2% sodium lauryl sulphate (e.g., at most about 2.0%, 1.8%, or 1.6%). In some instances, the oral care composition includes at least about 0.4% cocamidopropyl betaine (e.g., at least about 0.5%, 0.6%, 0.7%, or 0.8%) and at most about 1.2% cocamidopropyl betaine (e.g., at most about 1.0%, or 0.8%). In some instances, the oral care composition includes about 0.4% D-glucopyranoside C10-C16 alkyl oligomeric (e.g., at least about 0.5%, 0.6%, 0.7%, or 0.8%) and at most about 1.2% D-glucopyranoside C10-C16 alkyl oligomeric (e.g., at most about 1.0% or 0.8%). For example, in some instances, the oral care composition includes about 1.57% sodium lauryl sulphate, about 0.70% cocamidopropyl betaine, and about 0.51% D-glucopyranoside C10-C16 alkyl oligomeric. In general, an oral care composition can include, for a three surfactant blend of sodium lauryl sulphate, cocamidopropyl betaine, D-glucopyranoside C10-C16 alkyl oligomeric, a ratio of surfactant components of about 3:0.5:0.5 respectively. For a two surfactant blend such as a blend of sodium lauryl sulphate and cocamidopropyl betaine, the oral care composition can include a ratio of surfactants of about 3:1.

While sodium lauryl sulphate, cocamidopropyl betaine, and D-glucopyranoside C10-C16 alkyl oligomeric have been described above as surfactants and detergents, other surfactants and detergents are also envisioned. Surfactants generally include anionic, nonionic, cationic and zwitterionic or amphoteric compositions. Examples of surfactants include soaps, sulfates (e.g., sodium lauryl sulfate and sodium dodecyl benzene sulfonate), sodium lauryl sarcosinate, sorbitan esters of fatty acids, sulfobetaines (e.g., cocamidopropylbatine), and D-glucopyranoside C₁₀₋₁₆ alkyl oligomeric. In general, surfactants are present in an amount from about 0.2 to about 8% by weight (e.g., from about 1 to about 5% or from about 1.5 to about 3.5%).

Thickening Agents

In some instances, the oral care compositions include a thickening agent. Examples of thickening agents include thickening silica (discussed above), polymers, clays, and combinations thereof. Applicants have discovered that thickening silica, for example, SILODENT 15 hydrated silica, in the amount between about 4% to about 8% by weight (e.g., about 6%) provide desirable in-mouth characteristics. The phrase “in-mouth characteritics” as described herein relates to the body and thickness of the composition as it foams in the mouth of a user.

Polishing Agents

In many instances, the oral care compositions include a polishing agent. In some instances, the polishing agent is an abrasive. Examples of polishing agents include carbonates (e.g., sodium bicarbonate, calcium carbonate) water-colloidal silica, precipitated silicas (e.g., hydrated silica), sodium aluminosilicates, silica grades containing alumina, hydrated alumina, dicalcium phosphates, insoluble sodium metaphosphate, and magnesiums (e.g., trimagnesium phosphate). A suitable amount of polishing agent is an amount that safely provides good polishing and cleaning and which, when combined with other ingredients in the oral care composition gives a smooth, flowable, and not excessively gritty composition. In general, when polishing agents are included in the oral care composition, they are present in an amount from about 5% to about 50% by weight (e.g., from about 5% to about 35%, or from about 7% to about 25%).

Carriers

In general, the oral care compositions include a carrier. Examples of carriers include water, polyethylene glycol, glycerin, polypropylene glycol, starches, sucrose, alcohols (e.g., methanol, ethanol, isopropanol, etc.), or combinations thereof. Examples of combinations include various water and alcohol combinations and various polyethylene glycol and polypropylene glycol combinations. In general, the amount of carrier in an oral care composition is determined based on the concentration of the binder system along with the amount of dissolved salts, surfactants, and dispersed phase.

Humectants

In many instances, the oral care compositions include a humectant. Generally, humectants are polyols. Examples of humectants include glycerin, sorbitol propyleneglycol, xylitol, lactitol, polypropylene glycol, polyethylene glycol, hydrogenated corn syrup and mixtures thereof. In general, the humectants can include from about 10% to about 60% by weight of the oral care composition.

Buffers and/or Salts

In many instances the oral care composition includes one or more buffers or salts. Examples of buffers and salts include primary, secondary, or tertiary alkali metal phosphates, citric acid, sodium citrate, sodium saccharin, tetrasodium pyrophosphate, sodium hydroxide, and the like.

Active Ingredients

Oral care compositions can be put to a variety of uses, for example, to prevent cavities, to whiten teeth, to freshen breath, to deliver oral medication, and to provide other therapeutic and cosmetic benefits. Accordingly, the oral care compositions can include one or more of a variety of active ingredients. Examples of active ingredients include the following: anti-caries agents (e.g., water soluble fluoride salts, fluorosilicates, fluorozirconates, fluorostannites, fluoroborates, fluorotitanates, fluorogermanates, mixed halides and casine); anti-tarter agents; anti-calculus agents (e.g. alkali-metal pyrophosphates, hypophosphite-containing polymers, organic phosphocitrates, phosphocitrates, polyphosphates); anti-bacterial agents (e.g., bacteriocins, antibodies, enzymes); anti-bacterial enhancing agents; anti-microbial agents (e.g., Triclosan, chlorhexidine, copper-, zinc- and stannous salts such as zinc citrate, zinc sulfate, zinc glycinate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenbis-(4-chloro-6-bromophenol)); desensitizing agents (e.g., potassium citrate, potassium chloride, potassium tartrate, potassium bicarbonate, potassium oxalate, potassium nitrate and strontium salts); whitening agents (e.g., bleaching agents such as peroxy compounds, e.g. potassium peroxydiphosphate); anti-plaque agents; gum protecting agents (e.g., vegetable oils such as sunflower oil, rape seed oil, soybean oil and safflower oil, and other oils such as silicone oils and hydrocarbon oils). The gum protection agent may be an agent capable of improving the permeability barrier of the gums. Other active ingredients include wound healing agents (e.g., urea, allantoin, panthenol, alkali metal thiocyanates, chamomile-based actives and acetylsalicylic acid derivatives, ibuprofen, flurbiprofen, aspirin, indomethacin etc.); tooth buffering agents; reminieralization agents; anti-inflammatory agents; anti-malodor agent; breath freashing agents; and agents for the treatment of oral conditions such as gingivitis or periodontitis.

Other Ingredients

In some instances, the oral care compositions include effervescing systems such as sodium bicarbonate citric acid systems, or color change systems.

The oral care compositions may also include one or more of the following: phenolic compounds (e.g., phenol and its homologues, including 2-methyl-phenol, 3-methyl-phenol. 4-methyl-phenol, 4-ethyl-phenol, 2,4-dimethol-phenol, and 3,4-dimethol-phenol); sweetening agents (e.g., sodium saccharin, sodium cyclamate, sucrose, lactose, maltose, and fructose); flavors (e.g., peppermint oil, spearmint oil, eucalyptus oil, aniseed oil, fennel oil, caraway oil, methyl acetate, cinnamaldehyde, anethol, vanillin, thymol and other natural or nature-identical essential oils or synthetic flavors); preservatives (e.g., p-hydroxybenzoic acid methyl, ethyl, or propyl ester, sodium sorbate, sodium benzoate, bromochlorophene, triclosan, hexetidine, phenyl silicylate, biguanides, and peroxides); opacifying and coloring agents such as titanium dioxide or FD&C dyes; and vitamins such as retinol, tocopherol or ascorbic acid.

Rheological Properties

The shear-thinning properties and other Theological properties of an oral care composition may be evaluated, for example, by measuring the yield point and shear slope of the composition, and/or by measuring the Newtonian viscosity of the composition. In one instance, the rheological properties of the oral care composition for shear slopes from 0-0.4 Pa/sec are defined by a yield point of y Pa and a shear slope of x Pa/sec, with the ratio of yield point y to shear slope x for formulations being less than y/(74−370x+470x²). The rheological properties of the oral care composition may also be defined by a Newtonian viscosity, for example, between 50 and 400 Pa of less than 1.0 Pa.s. In some instances, where the yeild point is less than 5 Pa, the shear slope is greater than 0.2 Pa/sec.

The rheological characteristics (e.g., flow characteristics) can be measured, for example, using a Rhometrics controlled stress rheometer. For example, measurements can be made at a constant temperature of 20° C. using a cone and plate geometry. The cone is 45 mm in diameter with a gap setting of 0.051 mm. Yield stress and shear slope are measured, and a flow curve measuring formulation viscosity is determined as a function of the applied stress. From the curve information concerning the thickness of the product and the strength of the structure at rest (zero shear viscosity and yield stress) the ease at which the structure will break down (i.e., shear thinning) can be determined. Additionally, creep measurements can be made to determine the Newtonian viscosity of the compositions at 50 Pa and 400 Pa.

Dispensing Toothbrushes

The oral care compositions may be used in dispensing toothbrushes, for example those described in copending U.S. applications: “ORAL CARE DEVICE”, Attorney Docket No. 00216-629001, filed Jun. 3, 2004; “ORAL CARE DEVICE”, Attorney Docket No. 00216-640001, and filed Jun. 3, 2004; “ORAL CARE DEVICE”, Attorney Docket No. 00216-641001, filed Jun. 3, 2004, the entire disclosure of which is incorporated by reference herein. Such toothbrushes include mechanical pumps, e.g., pumps that operate by peristaltic action.

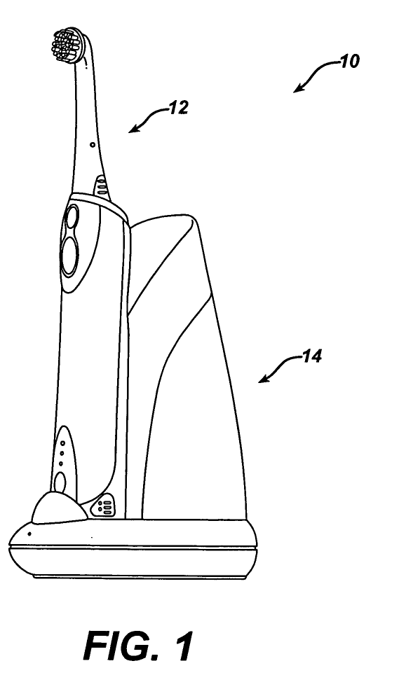

A suitable toothbrush that is described in the above-referenced application is shown in FIGS. 1-3B herein, and described below.

Referring to FIG. 1, an embodiment of an oral care system 10 is shown that includes an oral care device 12, in this case a toothbrush, and a docking station 14 that holds the oral care device 12 in an upright position within a receiving portion of the docking station. As will be described in much greater detail below, oral care device 12 is a power toothbrush having a motorized head and is designed to discharge a fluid, such as a dentifrice or mouthwash or a combination of various fluids, during the brushing cycle. The docking station 14 is designed to recharge batteries that are located within the oral care device, and to refill the oral care device with the fluid(s).

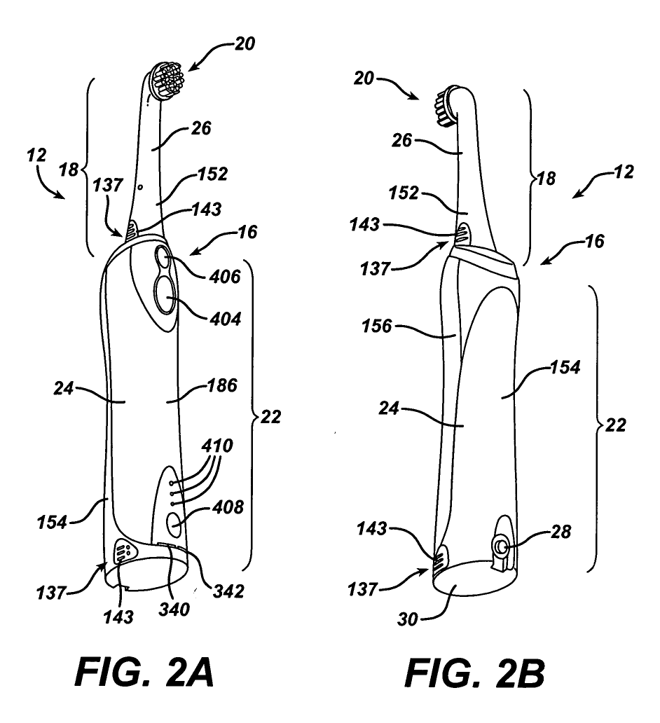

Turning to FIGS. 2A and 2B, oral care device 12 includes a multi-component, separable housing 16 consisting of three interconnected components 152, 154 and 156. As assembled, the oral care device 12 includes a distal portion 18 at which a head 20 is located and a proximal portion 22 at which a handle 24 is located. Connecting handle 24 and head 20 is neck 26. Head 20 is sized to fit within a user's mouth for brushing, while the handle 24 is graspable by a user and facilitates manipulation of the head 20 during use.

Referring to FIG. 2B, showing a rear view of the oral care device 12, an inlet 28 is positioned near an end surface 30 at the proximal portion 22 of the oral care device. As will be described in greater detail below, the inlet 28 is matable with an outlet located at the docking station 14 for refilling a fluid path within component 154. By positioning the inlet 28 distal of the end surface 30, the inlet is spaced above a seating surface within the receiving portion of the docking station where substances (e.g., dentifrice, water, dust) may accumulate, so that substances will not interfere with mating between the inlet 28 and the outlet.

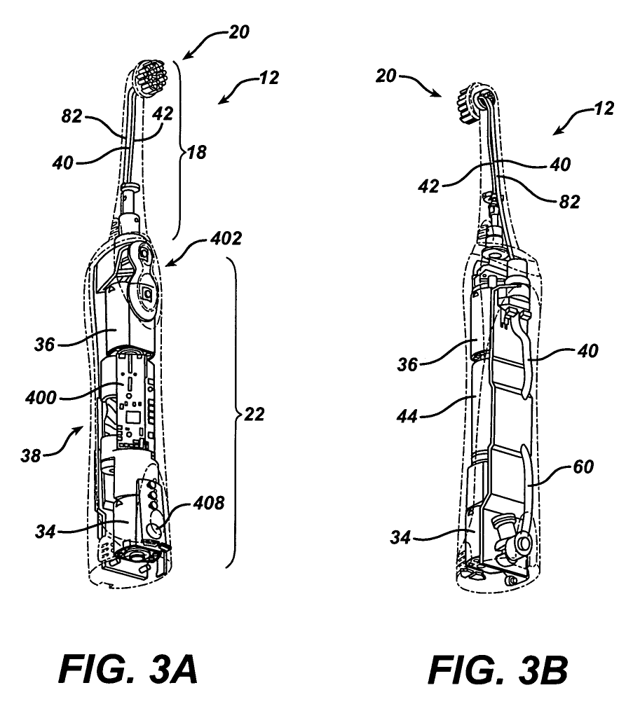

Referring now to FIGS. 3A and 3B, internal components of the oral care device 12 are shown. Oral care device 12 includes motors 34 and 36. Motor 34 drives a pumping assembly 38, that is used to transfer a fluid along a fluid passageway 40 (see FIG. 3B) toward the distal portion 18 of the oral care device 12. Pumping assembly 38 transfers fluid by compressing a portion of tube 60 with a compression element. In some embodiments, motor 34 is reversible and can move fluid in an opposite direction, toward the proximal portion 22 of the oral care device 12. Moving the fluid in the opposite direction may, for example, reduce or, in some cases, even eliminate any leaking of fluid from the head that may occur due to pressure build-up within the passageway. Motor 36 drives a drive shaft 42, which in turn moves (e.g., rotates) the head 20. To supply power to motors 34, 36, a rechargeable battery 44 is electrically coupled to the motors. A suitable rechargeable battery is a Li Ion UR 14500P, available from Sanyo.

### EXAMPLES

### Example 1

The dentifrice formulation shown in Table 3 is an example of a dentifrice having desirable rheological properties for dispensing through a dispensing toothbrush. The dispensing dentifrice has a 0 shear viscosity of 2.05E+04 Pa×s, a yield point of 18.85 Pa, and a shear slope of 0.104 Pa/sec. Additionally, the dispensing dentifrice has a Newtonian viscosity at 50 Pa of 1.45E+01 Pa×s and a Newtonian viscosity at 400 Pa of 0.00E+00 Pa×s. All measurements were taken using the Rheometrics controlled stress rheometer as described above.

TABLE 3
Formulation of Dispensing Dentifrice

| Raw Material | Concentration (% by wt.) |
|---|---|
| Hydrous Sodium Lithium Magnesium Silicate | 0.50 |
| Sodium Carboxymethylcellulose | 0.38 |
| Whitening Silica | 10 |
| Thickening Silica | 6 |
| Glycerine | 10 |
| Sorbitol | 30 |
| Sodium Saccharin | 0.30 |
| Tetrasodiumpyrophosphate | 0.025 |
| Sodium Hydroxide | 0.0625 |
| Sodium Lauryl Sulphate | 1.57 |
| Cocamidopropyl Betaine | 0.70 |
| D-Glucopyranoside, C10-16 Alkyl, Oligomeric | 0.51 |
| Flavor | 1.00 |
| Purified Water | 38.9525 |

Additional examples of dentifrice formulations are provided in Tables 4-7 below.

The oral care compositions described in Tables 4-7 are made as described in a two phase system as described.

Phase A: Approximately ¾ of the water is added to a kettle, with the remaining portion retained for Phase B. Sorbitol is added to the water and mixed thoroughly, and the mixture is meated to 80° C. Once the water has reached 80° C. the laponite D is slowly added and mixed for 15 minutes. The CMC is then added to glycerine and mixed until a uniform dispersion is formed. The CMC and glycerine blend is then added to the kettle and mixed for 30 minutes. Salts are then added (i.e., Saccharin, tetrasodiumpyrophosphate, and sodium hydroxide) to the kettle and mixed until blended. Once the above three ingredients are blended, the kettle is removed and cooled, at which point the whitening silica is added. Once the addition is complete the composition is mixed for min 30 minutes or until uniformly blended.

Phase B: Using remaining water from above, the surfactants (SLS, Cocamidopropyl Betaine, and D-Glucopyranoside, C10-16 Alkyl, Oligomeric) are added and mixed for 30 minutes or until blended. The thickening silica is then added and mixed for 15 minutes or until uniformly dispersed. After the thickening silica is uniformly dispersed, the flavor is added and mixed for 10 minutes or until uniformly blended. Once the kettle temperature is below 40° C., phase B is added to the kettle containing phase A and mixed for minimum of 30 minutes or until uniformly blended.

TABLE 4
Dentifrice formulations

| Raw material | | | | |
|---|---|---|---|---|
| DI Water | 38.80% | 38.95% | 38.70% | 39.51% |
| Hydrous Sodium Lithium | | 0.50% | 0.50% | 0.50% |
| Magnesium Silicate | | | | |
| Sodium carboxymethylcellulose | | 0.38% | 0.38% | 0.38% |
| Xanthan gum | | | | |
| Carrageenan Benvisco LB-2105 | 0.75% | | | |
| Abrasive Silica | 10% | 10% | 10% | 10% |
| Thickening Silica | 6% | 6% | 6% | 6% |
| Sodium Fluoride | 0.25% | | 0.25% | 0.25% |
| Glycerine | 10% | 10% | 10% | 10% |
| Sorbitol | 30% | 30% | 30% | 30% |
| Sodium Saccharin | 0.30% | 0.30% | 0.30% | 0.30% |
| Tetrasodiumpyrophosphate | 0.03% | 0.03% | 0.03% | 0.03% |
| Sodium Hydroxide | 0.10% | 0.06% | 0.06% | 0.06% |
| Sodium Lauryl Sulphate | 1.57% | 1.57% | 1.57% | 1.57% |
| Cocamidopropyl Betaine | 0.70% | 0.70% | 0.70% | 1.40% |
| D-Glucopyranoside, | 0.51% | 0.51% | 0.51% | |
| C10-16 Alkyl, Oligomeric | | | | |
| Phorphoric Acid | | | | |
| Monobasic Sodium Phosphate | | | | |
| (USP)(Anhydrous) | | | | |
| Disodium Edetate USP | | | | |
| Hydrogen Peroxide 35% | | | | |
| (as supplied) | | | | |
| Calcium Peroxide | | | | |
| Nipagin M | | | | |
| Niipasol M | | | | |
| Glycosperse L-20 | | | | |
| Flavor | 1.00% | 1.00% | 1.00% | |

TABLE 5
Dentifrice formulations

| Raw material | | | | |
|---|---|---|---|---|
| DI Water | 39.89% | 40.48% | 30.92% | 36.92% |
| Hydrous Sodium Lithium | 0.50% | 0.50% | | 0.05% |
| Magnesium Silicate | | | | |
| Sodium carboxymethylcellulose | 0.38% | 0.38% | | 0.38% |
| Xanthan gum | | | | |
| Carrageenan Benvisco LB-2105 | | | 0.75% | |
| Abrasive Silica | 10% | 10% | 10% | 10% |
| Thickening Silica | 6% | 6% | 6% | 6% |
| Sodium Fluoride | 0.25% | 0.25% | 0.25% | 0.25% |
| Glycerine | 10% | 10% | 10% | 10% |
| Sorbitol | 30% | 30% | 30% | 30% |
| Sodium Saccharin | 0.30% | 0.30% | 0.30% | 0.30% |
| Tetrasodiumpyrophosphate | 0.03% | 0.03% | | 3.00% |
| Sodium Hydroxide | 0.06% | 0.06% | | 0.10% |
| Sodium Lauryl Sulphate | 1.57% | 2.00% | 2.00% | 2.00% |
| Cocamidopropyl Betaine | | | | |
| D-Glucopyranoside, | 1.02% | | | |
| C10-16 Alkyl, Oligomeric | | | | |
| Phorphoric Acid | | | 0.20% | |
| Monobasic Sodium Phosphate | | | 0.10% | |
| (USP)(Anhydrous) | | | | |
| Disodium Edetate USP | | | 0.03% | |
| Hydrogen Peroxide 35% | | | 8.45% | |
| (as supplied) | | | | |
| Calcium Peroxide | | | | |
| Flavor | | | 1.00% | 1.00% |

TABLE 6
Dentifrice formulations

| Raw material | | | | |
|---|---|---|---|---|
| DI Water | 56.76% | 56.66% | 66.84% | 49.52% |
| Hydrous Sodium Lithium | 1.00% | 1.00% | 1.00% | |
| Magnesium Silicate | | | | |
| Sodium carboxymethylcellulose | | | | |
| Xanthan gum | 0.10% | 0.20% | 0.02% | |
| Carrageenan | | | | 0.75% |
| (BENVISCO LB-2105) | | | | |
| Abrasive Silica | 10.00% | 10.00% | | 7.00% |
| Thickening Silica | | | | 6.00% |
| Sodium Fluoride | | | | |
| Glycerine | 10.00% | 10.00% | 10.00% | 10.00% |
| Sorbitol | 20.00% | 20.00% | 20.00% | 20.00% |
| Sodium Saccharin | 0.04% | 0.04% | 0.04% | 0.30% |
| Tetrasodiumpyrophosphate | | | | |
| Sodium Hydroxide | | | | |
| Sodium Lauryl Sulphate | | | | 2.00% |
| Cocamidopropyl Betaine | | | | |
| D-Glucopyranoside, | | | | |
| C10-16 Alkyl, Oligomeric | | | | |
| Phorphoric Acid | | | | 0.20% |
| Monobasic Sodium Phosphate | | | | 0.20% |
| (USP)(Anhydrous) | | | | |
| Disodium Edetate USP | | | | 0.03% |
| Hydrogen Peroxide 35% | | | | |
| (as supplied) | | | | |
| Calcium Peroxide | | | | 3.00% |
| Methylparaben | 0.06% | 0.06% | 0.06% | |
| Propylparaben | 0.04% | 0.04% | 0.04% | |
| Polyoxylethylene 20 Sorbitan | 1.00% | 1.00% | 1.00% | |
| Monolurate | | | | |
| Flavor | 1.00% | 1.00% | 1.00% | 1.00% |

TABLE 7
Dentifrice formulations

| Raw material | | | | |
|---|---|---|---|---|
| DI Water | 36.95% | 40.95% | 40.33% | 40.96% |
| Hydrous Sodium Lithium | 0.50% | 0.50% | 0.75% | 0.50% |
| Magnesium Silicate | | | | |
| Sodium carboxymethylcellulose | 0.38% | 0.38% | 0.50% | 0.13% |
| Xanthan gum | | | | |
| Carrageenan Benvisco LB-2105 | | | | |
| Abrasive Silica | 10% | 10% | 10% | 10% |
| Thickening Silica | 8% | 4% | 4% | 4% |
| Sodium Fluoride | | | 0.25% | 0.25% |
| Glycerine | 10% | 10% | 10% | 10% |
| Sorbitol | 30% | 30% | 30% | 30% |
| Sodium Saccharin | 0.30% | 0.30% | 0.30% | 0.30% |
| Tetrasodiumpyrophosphate | 0.03% | 0.03% | 0.03% | 0.03% |
| Sodium Hydroxide | 0.06% | 0.06% | 0.06% | 0.06% |
| Sodium Lauryl Sulphate | 1.57% | 1.57% | 1.57% | 1.57% |
| Cocamidopropyl Betaine | 0.70% | 0.70% | 0.70% | 0.70% |
| D-Glucopyranoside, | 0.51% | 0.51% | 0.51% | 0.51% |
| C10-16 Alkyl, Oligomeric | | | | |
| Phorphoric Acid | | | | |
| Monobasic Sodium Phosphate | | | | |
| (USP)(Anhydrous) | | | | |
| Disodium Edetate USP | | | | |
| Hydrogen Peroxide 35% | | | | |
| (as supplied) | | | | |
| Calcium Peroxide | | | | |
| Nipagin M | | | | |
| Niipasol M | | | | |
| Glycosperse L-20 | | | | |
| Flavor | 1.00% | 1.00% | 1.00% | 1.00% |

All of the above formulations described in the above examples can be used in a dispensing brush system. The Laponite xanthan gum formulations described are also useful in a spray brush. Each of the formulations has been shown acceptable to consumer, and each of the formulations has been shown to be stable with time through accelerated aging studies.

The optimal rheology of the oral care composition will depend in part on the type of dispensing toothbrush used. For example, the thickness and shear-thinning characteristics may depend on the robustness of the mechanical pump, with more robust pumps tolerating higher viscosity and/or less shear-thinning compositions. A number of embodiments of the invention have been described. Nevertheless, it will be understood that various modifications may be made without departing from the spirit and scope of the invention. Accordingly, other embodiments are within the scope of the following claims.

1. An oral care composition comprising:
a carrier;
a binder; and
an active ingredient,
wherein the oral care composition exhibits a yield point of y Pa and a shear slope of x Pa/Sec, and wherein the ratio of yield point to shear slope is less than y/(74−370x+470x²), provided that when the yield point is less than 5 Pa, the shear slope is greater than 0.2 Pa/Sec.

2. The oral care composition of claim 1 wherein the yield point is greater than 5 Pa.

3. The oral care composition of claim 1 wherein the binder comprises at least one of hydrous sodium lithium magnesium silicate or sodium carboxymethylcellulose.

4. The oral care composition of claim 3 wherein the binder comprises about 0.1 to about 2.0% by weight of hydrous sodium lithium magnesium silicate or sodium carboxymethylcellulose.

5. The oral care composition of claim 3 wherein the binder comprises both hydrous sodium lithium magnesium silicate and sodium carboxymethylcellulose.

6. The oral care composition of claim 1 wherein the binder comprises a synthetic clay.

7. The oral care composition of claim 6 wherein the synthetic clay comprises a laponite, a hectorite, or a betaonite.

8. The oral care composition of claim 1 further comprising a surfactant or a detergent.

9. The oral care composition of claim 8 wherein the surfactant comprises at least one of sodium lauryl sulphate, cocamidopropyl betaine, or D-glucopyranoside C₁₀₋₁₆ alkyl oligomeric.

10. The oral care composition of claim 8 wherein the surfactant comprises sodium lauryl sulphate, cocamidopropyl betaine, and D-glucopyranoside C₁₀₋₁₆ alkyl oligomeric.

11. The oral care composition of claim 1 further comprising a thickening agent.

12. The oral care composition of claim 11 wherein the thickening agent comprises a thickening silica.

13. The oral care composition of claim 11 comprising between about 4% and about 8% thickening silica

14. The oral care composition of claim 13 comprising about 6% thickening silica.

15. The oral care composition of claim 11 wherein the thickening agent comprises a polymer.

16. The oral care composition of claim 15 wherein the polymer comprises an alginate, cellulose, a cellulose derivative, or carageenan.

17. The oral care composition of claim 16 wherein the polymer comprises carageenan.

18. The oral care composition of 17 comprising about 0.1 to about 2.0% by weight of carageenan.

19. The oral care composition of claim 1 comprising hydrous sodium lithium magnesium silicate, sodium carboxymethylcellulose, sodium lauryl sulphate, cocamidopropyl betaine, and D-glucopyranoside C₁₀₋₁₆ alkyl oligomeric.

20. The oral care composition of claim 1 comprising a polishing agent.

21. The oral care composition of claim 20 wherein the polishing agent comprises sodium bicarbonate, water-colloidal silica, percipiated silicas, sodium aluminosilicate, silica grades containing alumina, hydrated alumina, dicalcium phosphate, insoluable sodium metaphosphate, calcium carbonate, magnesium, trimagnesium phophate or combinations thereof.

22. The oral care composition of claim 20 wherein the polishing agent comprises abrasive silica or hydrated silica.

23. The oral care composition of claim 1 comprising xanthan gum and hydrous sodium lithium magnesium silicate.

24. The oral care composition of claim 23 comprising about 0.1 to about 2.0% by weight combined of xanthan gum and hydrous sodium lithium magnesium silicate.

25. The oral care composition of claim 23 wherein the ratio of the xanthan gum to hydrous sodium lithium magnesium silicate is about 0.5:1 to about 2:1.

26. The oral care composition of claim 1 wherein the active ingredient comprises an antimicrobial agent, a phenolic compound, an anti-inflammatory agent, an anti-caries agent, a plaque buffer, a desensitizing agent, an anti-calculus agent, a biomolecule, or combinations thereof.

27. The oral care composition of claim 1 wherein the carrier comprises water, polyethylene glycol, polypropylene glycol, glycerin or combinations thereof.

28. The oral care composition of claim 1 wherein the oral care composition has a Newtonian viscosity at 50 to 400 Pa of less than 1.0 Pa.s.

29. The oral care composition of claim 28 wherein the oral care composition has a Newtonian viscosity at 50 to 400 Pa of less than 0.5 Pa.s.

30. The oral care composition of claim 1 comprising a humectant.

31. The oral care composition of claim 30 wherein the humectant comprises glycerin, sorbitol or a combination thereof.

32. The oral care composition of claim 1 comprising a salt.

33. The oral care composition of claim 32 wherein the salt is selected from the group consisting of sodium saccharin, tetrasodium pyrophosphate, sodium hydroxide and combinations thereof.

34. The oral care composition of claim 1 wherein the oral care composition comprises a dentifrice.

35. An oral care composition comprising:
a carrier, and, dispersed in the carrier,
sodium lauryl sulphate;
cocamidopropyl betaine; and
D-glucopyranoside C₁₀₋₁₆ alkyl oligomeric.

36. The oral care composition of claim 35 further comprising hydrous sodium lithium magnesium silicate and sodium carboxymethylcellulose.

37. The oral care composition of claim 36 comprising:
about 0.1 to 1.0% hydrous sodium lithium magnesium silicate;
about 0.1 to 1.0% sodium carboxymethylcellulose;
about 1.0 to 2.0% sodium lauryl sulphate;
about 0.2 to 1.2% cocanidopropyl betaine; and
about 0.1 to 1.0% D-glucopyranoside C₁₀₋₁₆ alkyl oligomeric.

38. The oral care composition of claim 37 comprising:
about 0.4 to 0.6% hydrous sodium lithium magnesium silicate;
about 0.35 to about 0.40% sodium carboxymethylcellulose;
about 1.5 to 1.6% sodium lauryl sulphate;
about 0.6 to 0.8% cocamidopropyl betaine; and
about 0.4 to 0.6% D-glucopyranoside C₁₀₋₁₆ alkyl oligomeric.

39. The oral care composition of claim 35 further comprising a thickening agent.

40. The oral care composition of claim 39 wherein the thickening agent comprises thickening silica.

41. The oral care composition of claim 40 comprising about 4% to about 8% thickening silica.

42. The oral care composition of claim 41 comprising about 6% thickening silica.

43. The oral care composition of claim 39 wherein the thickening agent comprises a polymer.

44. The oral care composition of claim 43 wherein the polymer comprises an alginate, cellulose, a cellulose derivative, or carageenan.

45. The oral care composition of claim 35 further comprising an abrasive.

46. The oral care composition of claim 35 wherein the oral care composition comprises a dentifrice.

47. An oral care composition comprising:
a carrier;
a binder comprising at least one of hydrous sodium lithium magnesium silicate or sodium carboxymethylcellulose; and
an active ingredient,
wherein the oral care composition exhibits a yield point of y Pa and a shear slope of x Pa/Sec, and wherein the ratio of yield point to shear slope is less than y/(74−370x+470x²), provided that when the yield point is less than 5 Pa, the shear slope is greater than 0.2 Pa/Sec.

48. The oral care composition of claim 47 wherein the binder comprises both hydrous sodium lithium magnesium silicate and sodium carboxymethylcellulose.

49. An oral care device comprising:
a housing including, at a distal portion of the housing, a head dimensioned to fit within a user's mouth;
a compressible member defining at least a portion of a fluid path disposed within the housing;
a pumping assembly disposed within the housing and configured to compress the compressible member progressively along at least a portion of its length to transfer fluid along the fluid path toward an outlet at the distal portion of the housing; and
an oral care composition, disposed within theoral care device, wherein the oral care composition exhibits a yield point of y Pa, and a shear slope of x Pa/sec, and wherein the ratio of yield point to shear slope is less than y/(474−370x+470x²), provided that when the yeild point is less than 5, the shear slope is greater than 0.2 Pa/sec.

50. The oral care device of claim 49 wherein the oral care composition comprises a dentifrice.

51. An oral care device comprising:
a housing including, at a distal portion of the housing, a head dimensioned to fit within a user's mouth; and
a pumping assembly disposed within the housing and configured to transfer an oral care composition along a fluid path from a source containing the composition toward an outlet at the distal portion of the housing;
wherein the oral care composition exhibits a yield point of y Pa, and a shear slope of x Pa/sec, and wherein the ratio of yield point to shear slope is less than y/(474−370x+470x²), provided that when the yeild point is less than 5, the shear slope is greater than 0.2 Pa/sec.

52. A method comprising brushing teeth of a mammal with an oral care composition comprising a yield point of y Pa, and a shear slope of x Pa/sec, wherein the ratio of yield point to shear slope is less than y/(74−370x+470x²), provided that when the yeild point is less than 5, the shear slope is greater than 0.2 Pa/sec.

53. The method of claim 52 wherein the oral care composition comprises a dentifrice.
